SEQUENCE LISTING (18 sequences)

SEQ ID NO: 1 - DNA, 231 bp, synthetic construct
  misc_feature 1..231: note = Synthetic: rec-alpha-Btx (V31) Coding sequence
  source 1..231: mol_type = other DNA
atgggtattgtctgtcacactacggcaacgagtccgatcagcgcagttacgtgcccgccgggtgaaaacctgtgttatcgtaaaatgtggtgcgatgtgttttgtagctctcgcggtaaagtggttgaactgggttgcgcagcaacctgtccgagcaaaaaaccgtacgaagaagttacctgctgttctacggataaatgtaatccgcatccgaaacagcgtccgggttaa

SEQ ID NO: 2 - protein, 76 aa, synthetic construct
  REGION 1..76: note = Synthetic: rec-alpha-Btx (V31) Translated protein sequence
MGIVCHTTATSPISAVTCPPGENLCYRKMWCDVFCSSRGKVVELGCAATCPSKKPYEEVTCCSTDKCNPHPKQRPG

SEQ ID NO: 3 - DNA, 219 bp, synthetic construct
  misc_feature 1..219: note = Synthetic: rec-alpha-CTX Coding sequence
  source 1..219: mol_type = other DNA
atgatccgttgcttcatcaccccggacatcacctctaaagactgcccgaatggccacgtctgctacacgaaaacctggtgcgacgctttctgctctatccgtggtaaacgtgttgacctgggttgcgctgctacctgcccgaccgttaaaaccggtgttgacatccagtgctgctctaccgacaactgcaacccgttcccgacccgtaaacgtccgtaa

SEQ ID NO: 4 - protein, 72 aa, synthetic construct
  REGION 1..72: note = Synthetic: rec-alpha-CTX Translated protein sequence
MIRCFITPDITSKDCPNGHVCYTKTWCDAFCSIRGKRVDLGCAATCPTVKTGVDIQCCSTDNCNPFPTRKRP

SEQ ID NO: 5 - DNA, 204 bp, synthetic construct
  misc_feature 1..204: note = Synthetic: rec-kappa-Btx coding sequence
  source 1..204: mol_type = other DNA
atgcgtacctgtctgattagcccgtccagcaccccgcaaacctgtccgaatggtcaagatatttgttttctgaaggcccagtgtgataaattttgcagcattcgtggcccggtgatcgaacagggttgcgttgcgacctgtccgcaatttcgctctaactaccgttcactgctgtgctgtaccaccgacaactgtaatcattaa

SEQ ID NO: 6 - protein, 67 aa, synthetic construct
  REGION 1..67: note = Synthetic: rec-kappa-Btx Translated protein sequence
MRTCLISPSSTPQTCPNGQDICFLKAQCDKFCSIRGPVIEQGCVATCPQFRSNYRSLLCCTTDNCNH

SEQ ID NO: 7 - DNA, 204 bp, synthetic construct
  misc_feature 1..204: note = Synthetic: rec-MTalpha- Coding sequence
  source 1..204: mol_type = other DNA
atgctgacctgcgttacctccaaatctatcttcggcatcacgacggaaaactgcccggacggccagaacctgtgcttcaaaaagtggtattatctgaaccatcgttacagcgatattacgtggggttgcgcagcaacctgtccgaaaccgacgaacgtgcgcgaaaccatccactgctgtgaaaccgacaagtgcaatgaataa

SEQ ID NO: 8 - protein, 67 aa, synthetic construct
  REGION 1..67: note = Synthetic: rec-MTalpha- Translated protein sequence
MLTCVTSKSIFGITTENCPDGQNLCFKKWYYLNHRYSDITWGCAATCPKPTNVRETIHCCETDKCNE

SEQ ID NO: 9 - DNA, 198 bp, synthetic construct
  misc_feature 1..198: note = Synthetic: rec-Mambalgin-1 coding sequence
  source 1..198: mol_type = other DNA
atgaagagagaagctgaagccttaaagtgctatcaacacggtaaagtcgtaacctgccacagagacatgaagttctgctatcacaacacaggtatgccttttagaaatttgaagttgatattgcaaggttgttcttcatcctgctctgaaactgaaaacaataagtgctgctccaccgacagatgtaacaaaggttca

SEQ ID NO: 10 - DNA, 222 bp, synthetic construct
  misc_feature 1..222: note = Synthetic: rec-Hannalgesin Coding sequence
  source 1..222: mol_type = other DNA
atgacgaaatgctacgttaccccggatgttaaaagcgaaacctgcccggctggtcaagatatttgctacacggaaacctggtgcgatgcgtggtgcaccagccgtggcaaacgcgtcaacctgggttgcgcggccacgtgtccgattgtgaaaccgggcgttgaaatcaaatgctgctccaccgacaactgtaacccgttcccgacccgcaaacgcccgtaa

SEQ ID NO: 11 - protein, 73 aa, synthetic construct
  REGION 1..73: note = Synthetic: rec-Hannalgesin Translated protein sequence
MTKCYVTPDVKSETCPAGQDICYTETWCDAWCTSRGKRVNLGCAATCPIVKPGVEIKCCSTDNCNPFPTRKRP

SEQ ID NO: 12 - DNA, 270 bp, synthetic construct
  misc_feature 1..270: note = Synthetic: rec-mSlurp1 (recombinant mouse Slurp1) Coding sequence
  source 1..270: mol_type = other DNA
atgtttcgctgctatacctgtgaacaaccgacggctatcaactcatgtaaaaatatcgctcaatgtaaaatggaagacaccgcctgcaaaaccgtgctggaaacggttgaagcggcctttccgttcaaccattccccgatggtcacccgtagctgcagctctagttgtctggcaacggatccggacggcattggtgttgcgcacccggtgttctgctgtttccgtgacctgtgtaactctggttttccgggctttgtggcgggcctgtaa

SEQ ID NO: 13 - protein, 89 aa, synthetic construct
  REGION 1..89: note = Synthetic: rec-mSlurp1 (recombinant mouse Slurp1) Translated protein sequence
MFRCYTCEQPTAINSCKNIAQCKMEDTACKTVLETVEAAFPFNHSPMVTRSCSSSCLATDPDGIGVAHPVFCCFRDLCNSGFPGFVAGL

SEQ ID NO: 14 - DNA, 249 bp, synthetic construct
  misc_feature 1..249: note = Synthetic: rec-hSlurp1(recombinant human Slurp1) coding sequence
  source 1..249: mol_type = other DNA
atgctgaaatgctacacctgcaaagaaccgatgacctctgcttcttgccgtaccatcacccgttgcaaaccggaagacaccgcttgcatgaccaccctggttaccgttgaagctgaatacccgttcaaccagtctccggttgttacccgttcttgctcttcttcttgcgttgctaccgacccggactctatcggtgctgctcacctgatcttctgctgcttccgtgacctgtgcaactctgaactgtaa

SEQ ID NO: 15 - protein, 82 aa, synthetic construct
  REGION 1..82: note = Synthetic: rec-hSlurp1(recombinant human Slurp1) Translated protein sequence
MLKCYTCKEPMTSASCRTITRCKPEDTACMTTLVTVEAEYPFNQSPVVTRSCSSSCVATDPDSIGAAHLIFCCFRDLCNSEL

SEQ ID NO: 16 - DNA, 255 bp, synthetic construct
  misc_feature 1..255: note = Synthetic: rec-mPate-B(recombinant mouse Pate B) Coding sequence
  source 1..255: mol_type = other DNA
atgctgatctgcaactcttgcgaaaaatctcgtgactctcgttgcaccatgtctcagtctcgttgcgttgctaaaccgggtgaatcttgctctaccgtttctcacttcgttggtaccaaacacgtttactctaaacagatgtgctctccgcagtgcaaagaaaaacagctgaacaccggtaaaaaactgatctacatcatgttcggtgaaaaaaacctgatgaacttcctcgagcaccaccaccaccaccactga

SEQ ID NO: 17 - protein, 84 aa, synthetic construct
  REGION 1..84: note = Synthetic: rec-mPate-B(recombinant mouse Pate B) Translated protein sequence
MLICNSCEKSRDSRCTMSQSRCVAKPGESCSTVSHFVGTKHVYSKQMCSPQCKEKQLNTGKKLIYIMFGEKNLMNFLEHHHHHH

SEQ ID NO: 18 - protein, 66 aa, synthetic construct
  REGION 1..66: note = Synthetic: rec-Mambalgin-1 Translated protein sequence
MKREAEALKCYQHGKVVTCHRDMKFCYHNTGMPFRNLKLILQGCSSSCSETENNKCCSTDRCNKGS

(19) United States
(12) Patent Application Publication
(10) Pub. No.: US 2025/0122548 A1
(43) Pub. Date: Apr. 17, 2025

(54) METHODS FOR MAKING DISULFIDE-RICH PEPTIDES AND PROTEINS

(71) Applicant: UNIVERSITY OF SOUTHERN CALIFORNIA, Los Angeles, CA (US)

(72) Inventors: Jiang XU, Los Angeles, CA (US); Lin CHEN, Los Angeles, CA (US)

(21) Appl. No.: 18/728,630

(22) PCT Filed: Jan. 19, 2023
(86) PCT No.: PCT/US2023/011168

§ 371 Date: Jul. 12, 2024

Related U.S. Application Data
(60) Provisional application No. 63/300,892, filed on Jan. 19, 2022.

(51) Int. Cl. C12P 21/02; C07K 1/113; C07K 1/36; C07K 14/46

(52) U.S. Cl. CPC C12P 21/02; C07K 1/1136; C07K 1/36; C07K 14/46

(57) ABSTRACT

In alternative embodiments, provided are methods that are reliable and scalable for making disulfide bond rich peptides and proteins. In alternative embodiments, provided are oxidation refolding methods to produce disulfide bond rich peptides and proteins. In alternative embodiments, methods as provided herein can be used to make any disulfide bond-containing proteins, including but not limited to: three finger neurotoxin peptides (such as for example, rec-α-Bungarotoxin (rec-αBtx), rec-α-Cobratoxin (rec-αCTX), κ-Bungarotoxin (rec-κBtx), rec-MTa, rec-hannalgesin, rec-Mambalgin, rec-Slurp, rec-Pate), antibodies and antibody fragments (such as single chain antibody), extracellular domain of viral membrane proteins, cell surface receptors, other disulfide-bond rich toxin peptides (such as dendrotoxin, conotoxin) and the like.

### RELATED APPLICATIONS

This application is a national phase application claiming benefit of priority under 35 U.S.C. § 371 to Patent Convention Treaty (PCT) International Application serial number PCT/US2023/011168, filed Jan. 19, 2023, now pending, which claims the benefit of priority under 35 U.S.C. § 119(e) of U.S. Provisional Application No. 63/300,892, filed Jan. 19, 2022, now expired. The aforementioned application is applications are expressly incorporated herein by reference in their entirety and for all purposes. All publications, patents, patent applications cited herein are hereby expressly incorporated by reference for all purposes.

### STATEMENT OF GOVERNMENT INTEREST

This invention was made with government support under GM064642 awarded by the National Institutes of Health. The government has certain rights in the invention.

### TECHNICAL FIELD

This invention generally relates to protein biochemistry. In alternative embodiments, provided are methods that are reliable and scalable for making disulfide bond rich peptides and proteins. In alternative embodiments, provided are oxidation refolding methods to produce disulfide bond rich peptides and proteins. In alternative embodiments, methods as provided herein can be used to make any disulfide bond-containing proteins, including but not limited to: three finger neurotoxin peptides (such as for example, rec-α-Bungarotoxin (rec-αBtx), rec-α-Cobratoxin (rec-αCTX), κ-Bungarotoxin (rec-κBtx), rec-MTa, rec-hannalgesin, rec-Mambalgin, rec-Slurp, rec-Pate), antibodies and antibody fragments (such as single chain antibody), extracellular domain of viral membrane proteins, cell surface receptors, other disulfide-bond rich toxin peptides (such as dendrotoxin, conotoxin) and the like.

### BACKGROUND

Many proteins or small peptide contain sophisticated disulfide bonds, which drastically increase the stability of the structure. On the other hand, the presence of such bonds makes it very hard to obtain these proteins directly from Escherichia coli, or E. coli.

Snake venom three finger toxin peptides (or three finger proteins or peptides, or TFPs) are such a group of macromolecules that have interesting properties. Some of them can bind nicotinic acetylcholine receptors in the muscle (muscle type nAChR), some of them have analgesic effect, such as mambalgin and hannalgesin, some of them bind to receptors in the neurological system, such as MTa and κ-Bungarotoxin, which bind to α 2B-adrenoceptor and α₃β₂ nAChR, respectively.

The mammalian genome also encodes a group of three finger toxin-like proteins whose function little is known, such as Slurp, Lynx and Pate.

Due to the complex disulfide bonds system in these toxin peptides, it is usually impossible to obtain correctly folded three-finger toxin peptide directly from E. coli and not efficiently produced from other secretion expression systems, such as Pichia pastoris. There were successful attempts using chemical synthesis, such as muscarinic toxin MT7 and MT and Mambalgin, but this method suffers from the problem of sophisticated synthesizing steps, low productivity, and extremely high costs. As such, a universal, high yield production protocol capable of producing high quality three finger neurotoxins is needed.

### SUMMARY

In alternative embodiments, provided are methods for producing a disulfide-linked protein, comprising:
(a) providing an isolated or substantially isolated bacterial inclusion body comprising a recombinantly expressed disulfide-linked protein or peptide recombinantly expressed in the bacteria.
wherein optionally the bacterial inclusion body is substantially isolated if about 50% to 99%, or about 60% to 98%, or about 70% to 95%, of non-bacterial components (for example, inclusion body components) or compositions have been removed;
(b) solubilizing the inclusion body in a solubilization buffer,
wherein optionally the solubilization buffer comprises urea and/or guanidine, and optionally the solubilization buffer comprises between about 5 to 9 M urea, or about 8 M urea, or between about 4 to 8 M guanidine, or about 6 M guanidine; and
(c) oxidatively refolding the inclusion body in a refolding buffer by reaction conditions comprising:
   (i) incubating the solubilized inclusion body in the refolding buffer optionally the incubating is for between about 1 to 24 hours, or for between about 6 to 12 hours,
   wherein optionally the incubating comprises conditions or, or is done under conditions of, between: about 2° C. to 8° C., or at about 4° C., or incubating in an ice-cold solution,
   and optionally the incubating comprises conditions or, or is done under conditions, comprising a pressurized reaction vessel, or under pressure in a compressed air or nitrogen tank or reaction vessel, and optionally the pressure during the incubating is between about 2 to 5 atmospheres,
   and optionally the refolding buffer is stirred, shaken or agitated during the incubating, and
   (ii) concentrating the incubated refolding buffer comprising the solubilized inclusion body and recombinantly expressed disulfide-linked protein or peptide,
   wherein optionally the concentrating comprises using a nanofiltration device,
   and optionally the concentrating comprises using a compressed nitrogen-gas- or air-driven ultrafiltration device, to generate a concentrated solution, optionally the incubated refolding buffer is concentrated to about 1, 2, 3, 4 or 5 ml, or is concentrated to a dry powder, or substantially dry powder, comprising the recombinantly expressed disulfide-linked protein or peptide,
   wherein optionally the concentrating comprises removing between about 80% to 99.5%, about 85% to 99%, or about 90% to 95%, of the water or liquid from the incubated refolding buffer,
   and optionally a substantially dry powder has between about 0.1% to 10%, or between about 0.5% and 5%, liquid content remaining, and
wherein optionally the refolding buffer comprises a physiologic saline or a buffered saline, optionally the refolding buffer is at about neutral pH or pH of about 7.4, or has a pH of between about 5 and 8,
and optionally the refolding buffer comprises a phosphate-buffered saline (PBS), or a water-based salt solution comprising disodium hydrogen phosphate, sodium chloride, potassium chloride, potassium dihydrogen phosphate or any combination thereof,
thereby producing a concentrated refolding buffer.

In alternative embodiments, the incubating comprises conditions comprising a temperature of about 4° C. or an ice-cold solution, and/or incubating under pressure, optionally by use of compressed air or by use of a nitrogen tank or a reaction vessel.

In alternative embodiments, provided are methods for producing and purifying, or substantially purifying or isolating, a disulfide-linked protein, comprising:
(a) providing an isolated or substantially isolated bacterial inclusion body comprising a recombinantly expressed disulfide-linked protein or peptide recombinantly expressed in the bacteria.
wherein optionally the bacterial inclusion body is substantially isolated if about 50% to 99%, or about 60% to 98%, or about 70% to 95%, of non-bacterial components (for example, inclusion body components) or compositions have been removed;
(b) solubilizing the inclusion body in a solubilization buffer,
wherein optionally the solubilization buffer comprises urea and/or guanidine, and optionally the solubilization buffer comprises between about 5 to 9 M urea, or about 8 M urea, or between about 4 to 8 M guanidine, or about 6 M guanidine;
(c) oxidatively refolding the inclusion body in a refolding buffer by reaction conditions comprising:
   (i) incubating the solubilized inclusion body in the refolding buffer optionally the incubating is for between about 1 to 24 hours, or for between about 6 to 12 hours,
   wherein optionally the incubating comprises conditions or, or is done under conditions of, between: about 2° C. to 8° C., or at about 4° C., or incubating in an ice-cold solution,
   and optionally the incubating comprises conditions or, or is done under conditions, comprising a pressurized reaction vessel, or under pressure in a compressed air or nitrogen tank or reaction vessel, and optionally the pressure during the incubating is between about 2 to 5 atmospheres,
   and optionally the refolding buffer is stirred, shaken or agitated during the incubating, and
   (ii) concentrating the incubated refolding buffer comprising the solubilized inclusion body and recombinantly expressed disulfide-linked protein or peptide,
   wherein optionally the concentrating comprises using a nanofiltration device,
   and optionally the concentrating comprises using a compressed nitrogen-gas- or air-driven ultrafiltration device, to generate a concentrated solution, optionally the incubated refolding buffer is concentrated to about 1, 2, 3, 4 or 5 ml, or is concentrated to a dry powder, or substantially dry powder, comprising the recombinantly expressed disulfide-linked protein or peptide,
   wherein optionally the concentrating comprises removing between about 80% to 99.5%, about 85% to 99%, or about 90% to 95%, of the water or liquid from the incubated refolding buffer,
   and optionally a substantially dry powder has between about 0.1% to 10%, or between about 0.5% and 5%, liquid content remaining, and
wherein optionally the refolding buffer comprises a physiologic saline or a buffered saline, optionally the refolding buffer is at about neutral pH or pH of about 7.4, or has a pH of between about 5 and 8,
and optionally the refolding buffer comprises a phosphate-buffered saline (PBS), or a water-based salt solution comprising disodium hydrogen phosphate, sodium chloride, potassium chloride, potassium dihydrogen phosphate or any combination thereof,
thereby producing a concentrated refolding buffer; and
(d) purifying or substantially purifying or substantially isolating the recombinantly expressed disulfide-linked protein or peptide from the concentrated refolding buffer by a method comprising:
   (i) re-solubilizing or solubilizing the concentrated refolding buffer, or the concentrated solution or dry powder comprising the recombinantly expressed disulfide-linked protein or peptide of step (d), by a method comprising adding the concentrated refolding buffer, or the concentrated solution or dry powder to a low ionic strength buffer before purifying or substantially purifying or substantially,
wherein optionally the low ionic strength buffer comprises a physiologic saline or a buffered saline, optionally the refolding buffer is at about neutral pH or pH of about 7.4, or has a pH of between about 5 and 8,
and optionally the low ionic strength buffer comprises a phosphate-buffered saline (PBS), or a water-based salt solution comprising disodium hydrogen phosphate, sodium chloride, potassium chloride, potassium dihydrogen phosphate or any combination thereof,
and optionally a low ionic strength buffer is a buffer having few salts, or is more dilute, than a physiologic saline; and
   (ii) purifying, isolating or substantially purifying or substantially isolating the disulfide-linked protein or peptide,
   wherein optionally the purifying, isolating or substantially purifying or substantially isolating is by a method comprising use of a chromatography, and optionally the chromatography comprises an affinity chromatography, an ion exchange chromatography, or a size exclusion chromatography,
   and optionally a substantially purified or substantially isolated disulfide-linked protein or peptide is between about 85% to 99.5% pure, or between about 90% to 99% pure.

In alternative embodiments of methods as provided herein:
the disulfide-linked protein or peptide comprises or is: a three-finger neurotoxin peptide, and optionally the three-finger neurotoxin peptide comprises or is: recombinant (rec)-α-Bungarotoxin (rec-αBtx), rec-α-Cobratoxin (rec-αCTX), κ-Bungarotoxin (rec-κBtx), rec-MTa, rec-hannalgesin, or rec-Mambalgin, rec-Slurp, rec-Pate);
the disulfide-linked protein or peptide comprises or is: an antibody, an antibody fragment, a single chain antibody, an extracellular domain of a viral membrane protein, a cell surface receptor, a dendrotoxin, or a conotoxin;
the bacterial inclusion body comprises or is or is derived from an inclusion body from a bacteria of the Escherichia genus, or is an Escherichia coli (E. coli), inclusion body; and/or
the solubilizing comprises use of a solubilization buffer comprising urea and/or guanidine, or comprising 50 mM Tris-HCl (pH 8.0), 8 M urea or 6 M guanidine-HCl and 5 mM 2-Mercaptoethanol (2-ME); or, 50 mM Tris-HCl (pH 9.0), 6 M guanidine-HCl, 5 mM 2-ME.

In alternative embodiments, provided are kits or products of manufacture comprising all materials, reagents and ingredients needed to practice methods as provided herein, and optionally also comprising instructions for practicing methods as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

FIG. 1 schematically illustrates an exemplary procedure for obtaining bioactive recombinant three finger toxins (TFTs) using E. coli.

FIG. 2 schematically illustrates an exemplary procedure, and instrument setup, for practicing an exemplary method as provided herein.

FIG. 3A illustrates an image of an SDS-PAGE gel separating recombinant (rec)-α-Bungarotoxin (rec-αBtx) protein as isolated using an exemplary methods as provided herein.

FIG. 3B illustrates a schematic of the structure of rec-αBtx protein.

FIG. 4 illustrates Table 1, showing a summary of statistics for the data and structures of six (6) recombinant three-fingered proteins' (rTFPs') structures that were solved with x-ray crystal diffraction data using molecular replacement with known homologous structures, as discussed in further detail, below.

FIG. 5 illustrates an SDS-PAGE image, illustrating a refolding condition screening of rTNFs; refolding product from each refolding condition were concentrated and analyzed by 15% non-reducing SDS-PAGE, as discussed in further detail, below.

FIG. 6A illustrates an SDS-PAGE image of rTFPs tested for their stability by prolonged storage at 4° C., as discussed in further detail, below.

FIG. 6B illustrates an SDS-PAGE image (lower image) and graphically illustrates data (upper image) showing that ultrafiltration of a refolded product to dry dramatically increased the purity for some rTFPs, as discussed in further detail, below.

FIG. 7A graphically illustrates data comparing the behavior of rec-αBtx, rec-κBtx, rec-mPate B in a gel filtration column, as discussed in further detail, below.

FIG. 7B illustrates an SDS-PAGE image where binding specificities of recombinant three-fingered proteins (rTFPs) were tested, as discussed in further detail, below.

FIG. 7C illustrates an SDS-PAGE image illustrating data showing that hannalgesin binds the extracellular domain of al subunit of the nicotinic acetylcholine receptor (α1ECD), like native αCTX isolated from Naja Kaouthia, as discussed in further detail, below.

FIG. 8A-B illustrate: microscopic FIG. 8A and fluorescence microscopic FIG. 8B images of rec-mPate B labeled with NHS-rhodamine and visualized the binding of rec-mPate B to spermatozoa freshly isolated from the epididymis of the mouse under the fluorescence microscope, as discussed in further detail, below.

FIG. 9A-F illustrate polarized light images under a microscope where six (6) recombinant three-fingered proteins' (rTFPs') structures were solved with x-ray crystal diffraction data using molecular replacement with known homologous structures; and the statistics for the data and structures are summarized in Table 1, as discussed in further detail, below.

Like reference symbols in the various drawings indicate like elements.

### DETAILED DESCRIPTION

In alternative embodiments, provided are methods that are reliable and scalable for making disulfide bond rich peptides and proteins. In alternative embodiments, provided are oxidation refolding methods to produce disulfide bond rich peptides and proteins. In alternative embodiments, methods as provided herein can be used to make any disulfide bond-containing proteins, including but not limited to: three finger neurotoxin peptides (such as for example, rec-α-Bungarotoxin (rec-αBtx), rec-α-Cobratoxin (rec-αCTX), κ-Bungarotoxin (rec-κBtx), rec-MTa, rec-hannalgesin, rec-Mambalgin, rec-Slurp, rec-Pate), antibodies and antibody fragments (such as single chain antibody), extracellular domain of viral membrane proteins, cell surface receptors, other disulfide-bond rich toxin peptides (such as dendrotoxin, conotoxin) and the like, which have wide applications.

The method is exemplified in producing recombinant three finger neurotoxin peptides from E. coli. Traditionally, such three finger neurotoxin peptides were purified from snake venoms, which is sophisticated and generally limited by the scarce source of snake venoms and hence very expensive. The recombinant three finger neurotoxin peptides produced by methods as provided herein were confirmed by either x-ray diffraction structural analysis and or activity test using either in vitro binding assay with known receptors, or by using immunofluorescent staining of known targets on live cells. In alternative embodiments, methods as provided herein provided an attractive alternative source of the disulfide bond rich peptides and proteins, including three finger neurotoxin peptides, which have great application potential both scientifically and commercially.

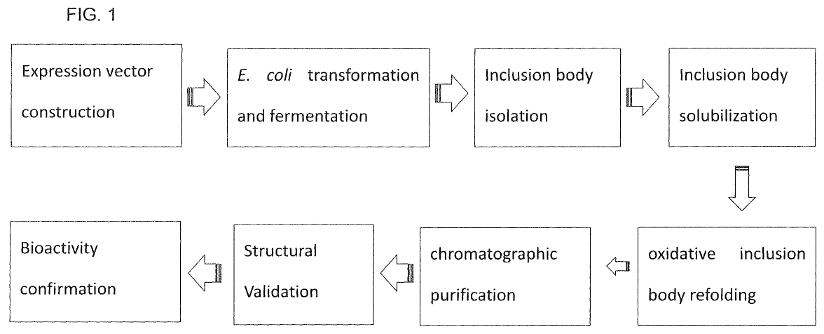

FIG. 1 schematically illustrates an exemplary procedure for obtaining bioactive recombinant three finger toxins using E. coli, comprising: expression vector construction; bacterial transformation and fermentation; inclusion body isolation; inclusion body solubilization; oxidative inclusion body refolding; chromatographic purification; structural validation and bioactivity confirmation. In alternative embodiments, prokaryotic expression vector construction, E. coli transformation, inclusion body isolation, solubilization, chromatographic purification, x-ray diffraction protein crystal structure determination follows general established protocols, see for example FIG. 1.

In alternative embodiments, methods as provided herein have at least three unique aspects:

First, the toxin peptides are expressed without any tag, thus significantly simplifies the purification procedure.

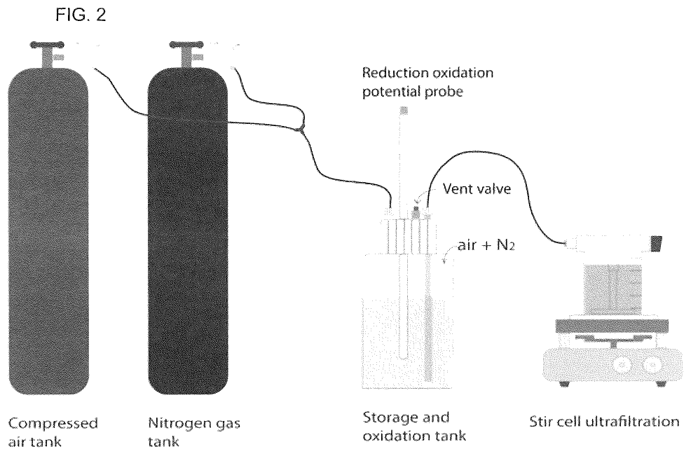

Second, for inclusion body oxidative refolding, we developed a unique protocol using a custom designed oxidation chamber, which also functions as a storage tank (as illustrated in FIG. 2) in which a mixture of compressed air (or pure O₂) and N₂ gas was used to drive the ultrafiltration device and to enhance the dissolved oxygen level in the refolding solution. The oxygen gas ratio is adjustable and the redox potential in the solution is monitored for optimized refolding result.

Third, no redox-pairs such as reduced-oxidized glutathione, or cysteine-cystine are used, only cysteine concentrations are optimized.

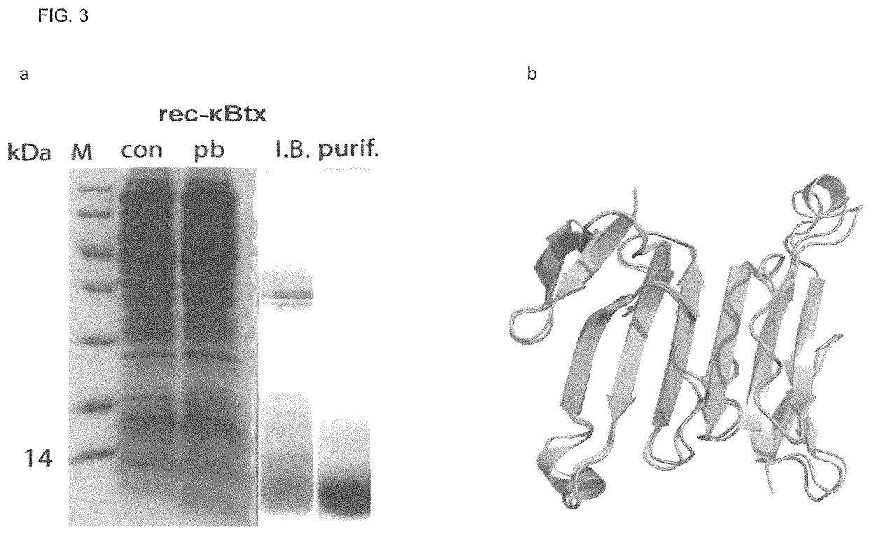

Using exemplary methods as provided herein, we have successfully produced recombinant three finger neurotoxin peptides such as rec-α-Bungarotoxin (rec-αBtx), rec-α-Cobratoxin (rec-αCTX), κ-Bungarotoxin (rec-κBtx), rec-MTa, rec-hannalgesin, rec-Mambalgin, rec-Slurp, rec-Pate, and the like, with five of them being structurally validated. As an example, shown here, the structural alignment of natural κBtx aligned with rec-κBtx shows the two aligned perfectly (see FIG. 3), which confirmed the method is robust and the product is authentic. These results indicate our method is generally applicable to a wide range of three finger toxins, which can be a competitive source to their natural counterpart.

In alternative embodiments, methods as provided herein include use of a specially designed oxidation chamber where the redox potential is monitored, which enabled refolding condition being closely monitored, thus drastically improved the reproducibility of the process and the quality of final product.

In alternative embodiments, methods as provided herein have are carried out in general molecular biology lab and can be scaled up easily. Given the importance of these toxin peptides in biomedical research, methods as provided herein are a significant step-forward in the field.

Finally, our method should in principle be applied to refold other disulfide bond rich proteins, which should be of general interest both scientifically and commercially.

Example Result is illustrated in FIG. 3A-B:

FIG. 3A show an image of an SDS-PAGE result of expression, isolation of inclusion body (I.B.), refolding of I.B. and purification of correctly folded rec-κBtx (con: uninduced E. coli cells, pb: induced E. coli cells, purif.: purified rec-κBtx);

FIG. 3B schematically illustrates a structural alignment of a known κBtx structure (green) with that of the rec-κBtx (violet).

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all techniques are carried out according to standard protocols.

### Example 1: Recombinant Three-Finger Proteins Out of the Lab: From Inclusion Bodies to High Quality Molecular Probes

This example demonstrates that methods as provided herein are effective and can be used to product disulfide bonded peptides and proteins.

Provided herein is a working pipeline for expression, purification of disulfide-bond rich three-finger neurotoxin peptides of snake venom origin, or their homologous protein of mammalian origin, using E. coli as the expression host. With this pipeline, we have successfully obtained high quality recombinant α-Bungarotoxin, k-Bungarotoxin, Hannalgesin, Mambalgin, α-Cobratoxin, MTα, Slurp1, Pate B etc. Milligrams to hundreds of milligrams of recombinant three finger proteins can be obtained within weeks in the lab. The recombinant peptides showed specificity in binding assay and six of them were crystallized and their structures were validated using X-ray protein crystallography.

Our method is different from previous attempts in that, 1. The recombinant toxins were expressed without any fusion tags, thus significantly simplifying the purification procedure and dramatically increasing the quality and the yield. 2. For each toxin, a universal refolding screen protocol was applied to search for refolding conditions. 3. A unique oxidation refolding protocol was carried out to ensure complete disulfide bond formation. Due to the extremely high quality of the recombinant peptides and high yield, our method provides an attractive alternative source of three-finger toxins or toxin-like proteins to their natural counterpart.

### Materials and Methods

### Buffer Used

Lysis buffer: 50 mM Tris-HCl (pH 8.0), 150 mM NaCl, 1% Triton X-100, 10 mM 2-mercaptoethanol (5 liter for 200 g of bacteria cell pellets)

Solubilization buffer: 50 mM Tris-HCl (pH 9.0), 6 M guanidine-HCl (or 8 M urea), and 5 mM 2-mercaptoethanol (should be freshly prepared).

Refolding screen buffer (see Table 2, below).

Restriction enzymes were from Takara or New England Biolabs. All chemicals were from Sigma-Aldrich unless otherwise stated.

Vector Construction, E. coli Fermentation and Inclusion Body Extraction

Genes encoding the toxin proteins were codon-optimized and synthesized (Integrated DNA Technologies Inc) with NdeI site on the 5′ end and a termination codon (TAA or TAG) at the 3′ end just before the XhoI sites. The genes were inserted into the NdeI and XhoI sites of pET30b (Novagen) and the reconstructed expression vector was transformed into the expression host BL21 (DE3). E. coli cells were fermented either with a home-made 5-liter fermenter or a BIOFLO3000 BIOREACTOR™ (New Brunswick Scientific) and induced for protein expression by adding 0.8 mM IPTG at an Optical density of approximately 18 to 19 and fermented for additional 4 hrs. In alternative embodiments, about 160 g to 400 g of bacteria pellets (wet weight) could be obtained and stored at −20° C. as 50 grams aliquots. To obtain the inclusion bodies, 200 g of bacteria was thawed in 1 liter of lysis buffer supplemented with 2 mg chicken egg lysozyme per gram of bacteria pellets of was then added and mixed well using a bench-top homogenizer (KitchenAid). The mixture was incubated on ice for 1 hr and sheared with the homogenizer at top-speed for 60 s and cooled in the cold room for 15 min, the shearing process was repeated twice until the solution become less sticky, which was then centrifuged at 10,000 g/4° C./15 min. The supernatant was discarded, and the pellets were subjected to a new round of resuspension-shearing-centrifugation process until the pellet became compact. The pellets were finally resuspended in 1 to 2 liters of lysis buffer and aliquot to 20 to 40 50-ml conical tubes, pellet down by centrifugation at 8000 g/10° C./15 min, and stored at −20° C. until use.

### I.B. Solubilization and Refolding Screen.

To solubilize the I.B., a solubilization buffer containing 50 mM Tris-HCl (pH 8.0), 8 M urea or 6 M guanidine-HCl and 5 mM 2-Mercaptoethanol (2-ME) was used. The choice of the solubilization buffer was based on the solubilization effect and contaminating protein level. Taken α-Bungarotoxin for example, this toxin refolded poorly in the presence of contaminating proteins and its I.B. was solubilized well with a solubilization buffer containing 8 M urea. So, after solubilization with 50 mM Tris base, 8 M urea, and 5 mM 2-mercaptoethanol, and centrifuged for 28,000 g/10 min/4° C. to get rid of insoluble bacteria debris, the pH of the supernatant was adjusted to 8.5 with concentrated HCl solution and further absorbed with Q Sepharose FF media (2 ml of solution/ml of Q media) equilibrated with 50 mM Tris-HCl (pH 8.5), 8 M urea. Attention should be paid to avoid using high concentration of reducing chemical reagents (like 100 mM of 2-mercaptoethanol) at the solubilization stage, which will lead to low refolding efficiency.

After absorption, the I.B. was ready for refolding. For other toxins with higher expression level and more compact inclusion bodies, a solubilization buffer containing 50 mM Tris-HCl (pH 9.0), 6 M guanidine-HCl, 5 mM 2-ME was used.

Refolding condition was optimized with a screening protocol scouting for NaCl concentration (0 or 200 mM), l-cysteine concentration (0-16 mM), 1-arginine (0 or 0.5 M), and detergent, such as NDSB-201 (0 or 0.2 M), etc. Standard refolding trial was made by diluting 200 μl of I.B. solution into 10 ml of refolding screen solution.

After refolding, the solutions were left at 4° C. overnight (more than 24 hr), and concentrated each with Amicon Ultra-15 (Millipore, 3 kDa NMWL) ultrafiltration devices to less than 200 μl. The retention was centrifuged at 18,000 g/4° C. for 15 min and the supernatant analyzed with non-reducing SDS-PAGE. The rest of the concentrated solutions were each divided into three parts and dialyzed against low ionic strength buffer with various pH values, such as 20 mM NaAc (pH 5.0), 20 mM HEPES (pH 7.5, adjusted with NaOH), or 20 mM Tris-HCl (pH 8.0), using home-made micro-dialysis devices (Fiala et al., 2011). Finally, the dialyzed solution was centrifuged at 18,000 g/4° C. for 15 min. The supernatant was analyzed using non-reducing SDS-PAGE for quantifying different refolding species of monomer and multimers.

### Preparative Refolding of Recombinant Toxins

After the initial screen, an optimized refolding condition was usually determined, based on the yield of monomeric species on non-reducing SDS-PAGE. For preparative refolding, fresh I.B. solution was poured all in once into the ice-cold refolding solution which was stirred rapidly by a magnetic bar throughout the whole process, at a volume ratio of 1:50. The refolded solution was left static over one week at 4° C., and concentrated with a compressed nitrogen-gas (or air) driven ultrafiltration device (350 ml Amicon Stirred Cell, 3 kDa NMWL membrane, Millipore) (coupled with a storage tank in refolding recombinant κ-Bungarotoxin). Typically, the refolded solution was concentrated to a very small volume of several ml, or to ‘dry’, depending on the type of the toxins proteins being refolded, and then filled with refolded protein solution and proceeded to do the ultrafiltration again, this process is repeated back and forth until all the refolded solution was concentrated, which usually takes about 2-4 weeks, during which time cysteine in the solution gradually react to form white cystine crystalline and precipitated out, and could usually clog the ultrafiltration membrane at the end phase of concentration, making the process longer. For some toxins, like recombinant MTα (rec-MTα), Hannalgesin (rec-Hannagesin), mouse Pate B (rec-mPateB), κ-Bungarotoxin (rec-κBtx), α-Bungarotoxin (rec-αBtx), it is better to concentrate to ‘dry’, leaving no noticeable liquid in the ultrafiltration device, which could dramatically increased the purity and quality of the product. For other toxins we tried like recombinant mambalgin-1 (rec-Mambalgin-1), mouse and human Slurp1 (rec-mSlurp1 and rec-hSlurp1), concentrating to dry significantly lowered the final yield. So, trial experiments should be made at this point. The concentrated product was then re-solubilized with a low ionic strength buffer, which was pre-determined in the dialysis experiment. Normally, proteins with isoelectric point (pI) over 7 was re-solubilized in 30 to 50 ml of 50 mM NaAc (pH 5.0), while proteins with pI less than 7 was solubilized in 50 mM Tris-HCl (pH 8.0). The solution was then filtered with a 0.2 μm filter and applied to mono S 5 50 GL column or mono Q 5 50 GL column, based on the pI of the proteins.

Bound proteins were eluted with a linear gradient of NaCl to 1 M. The eluted peaks were again analyzed by non-reducing SDS-PAGE. Those eluted later usually contained contaminating proteins, or species inter-connected by intermolecular disulfide bonds.

For those not concentrated to ‘dry’ but to small volume, an additional dialysis step was usually added, in which the concentrated solution was dialyzed against the low-ionic strength buffer and applied to the ion exchange column. For the proteins we tried, a single, large peak was usually seen using the mono S column (See result), and several large peaks were seen using the mono Q column, in which the target species was usually contained in the first peak. At this stage, the refolded toxin was fairly pure, but for XRD experiments, gel filtration was usually done with a Superdex 75 10 300 GL column (GE Healthcare), to further increase the purity of the product and to buffer-exchange to 200 mM ammonium acetate (pH 7° C.).

### Native Gel Shift Assay

5 μg of HAP peptide (Harel et al., 2001; Kudryavtsev et al., 2020) were mixed with 5 μg of recombinant respectively, incubated at room temperature for 15, and run on a 15% native PAGE gel with 50 mM NaAc (pH 5.0) at 120 v/60 min/4° C. For the binding assay with the nicotinic acetylcholine receptors, 5 μg of rec-αCTX, rec-Hannalgesin, or α-Cobratoxin (αCtx) (Sigma-Aldrich, C6903) was mixed with 5 μg of recombinant the extracellular domain of the al subunit of muscle type nicotinic acetylcholine receptor (α1ECD) (Dellisanti et al., 2007; Yao et al., 2002), incubated on ice for 15 min and run on 12% native gel (standard discontinuous PAGE gel (without SDS), 6% for top layer and 10% for bottom layer with Tris-Glycine buffer (pH 8.3, without SDS) as the running buffer at 120 v/90 min/4° C. Gels were stained with coomassie brilliant blue as described (Wittig and Schägger, 2005).

Labeling of Rec-mPate B with Fluorescence Dye and Visualization of Binding of Rec-mPate B to the Mouse Spermatozoa

rec-mPate B was labeled with NHS-rhodamine according to the manufacturer's recommended protocol. Briefly, 25 μl of rec-mPate B solubilized in PBS (pH 7.4) at 27.2 mg/ml was mixed with 20 mM HEPES (pH 7), 4.13 μl of 18.9 mM NHS-Rhodamine DMSO solution (ThermoFisher) and incubated at room temperature for 60 min, and dialyzed exhaustively against 20 mM HEPES, 0.15 M NaCl. Mouse spermatozoa was obtained as described, and was mixed with 1:1000 dilution of the Rhodamine labeled rec-mPate B, washed three times with PBS, and observed under a laser confocal fluorescence microscope.

X-Ray Protein Crystal Diffraction Structural Validation of rTFP

Purified toxin proteins were concentrated to 15 to 150 mg/ml with Amicon Ultra-15 and Amicon Ultra-0.5 (3 kDa NMWL) tubes. Sitting drop crystal screening was done using a robotic system (Crystal Gryphon, Art Robbins Instrument). For crystallization of rec-αBtx, rec-αBtx was complexed with HAP peptide (Harel et al., 2001) by mixing at a molar ratio of 1:1.5, incubated at room temperature for 30 min and then diluted 100 fold with 20 mM NaAc, pH 5.0 and applied to mono S column. Bond protein was eluted with linear gradient of NaCl to 1 M and the sharp peak containing the rec-αBtx-HAP complex was collected, pooled and concentrated to about 13 mg/ml, dialyzed against 0.1 M HEPES (pH 7.0) exhaustively at 4° C.

For crystallization of other recombinant three-fingered proteins (rTFPs), purified rTFP were concentrated to about 80 to about 150 mg/ml and screened for crystal growth. Hanging drop method was then done manually to optimize the growth condition, by mixing equal volume of well solution and the toxin protein, and incubating both at 4° C. and 18° C.

Crystals were then harvested under cryo-conditions and X-ray diffraction data of for rec-kBtx·rec-mambalgin 1 and rec-αBtx-HAP complex were collected either with a RIGAKU MICROMAX™-007 home X-ray source coupled with an R-AXIS IV++ image plate. For rec-MTα, X-ray diffraction data was collected at ADVANCED PHOTON SOURCE™ (Argonne National Laboratory, Lemont, IL). The X-ray diffraction data of rec-Hannalgesin and rec-αCTX were collected at Advanced Light Source (Lawrence Berkeley National Laboratory, Berkeley, CA).

Data was processed with HKL2000™ (Otwinowski and Minor, 1997) or IMOSFLM™ (Battye et al., 2011), CCP4 suite (Winn et al., 2011), Molecular Replacement, structure build and refinement was done in PHENIX™ (Liebschner et al., 2019) and Coot (Liebschner et al., 2019).

### Results

Our Pipeline is Universally Applicable to a Wide Variety of TFPs with High Yield

Our idea is to use E. coli to produce high quality three-fingered proteins (TFPs) of biomedical interests. Our pipeline involved codon optimization of the encoding DNA sequence, recombinant protein expression in E. coli, isolation of I.B., refolding condition scouting, preparative refolding and purification, structural validation with x-ray diffraction and biochemical methods (see exemplary protocol of FIG. 1). From the known protein or encoding DNA sequences, production of a rTFP usually took about 4 to 5 weeks. For each of the rTFPs, a non-reducing SDS-PAGE was carried out to check the purity of the final product and quantify species with inter-molecule disulfide bonds. For a couple of TFPs of various origin (see Table 2, below), our pipeline was shown to be robust and successful).

It is hard to imagine expressing, refolding rTFP of several kDa using E. coli and achieving milligrams to hundreds of milligrams in a common molecular biology lab. However, with our pipeline, we obtained over one hundred milligrams of rec-MTα, rec-Hannalgesin, rec-αCTX and rec-mPate B, tens of milligrams of rec-mambalgin-1, Slurp1 and milligrams of rec-kBtx and rec-αBtx with only one round of experiment (usually finished within approximately 4 to 5 weeks), which to our knowledge, has never been reported before.

Most Useful Scouting Conditions for Refolding rTFP are Cysteine and Salt Concentration, and pH

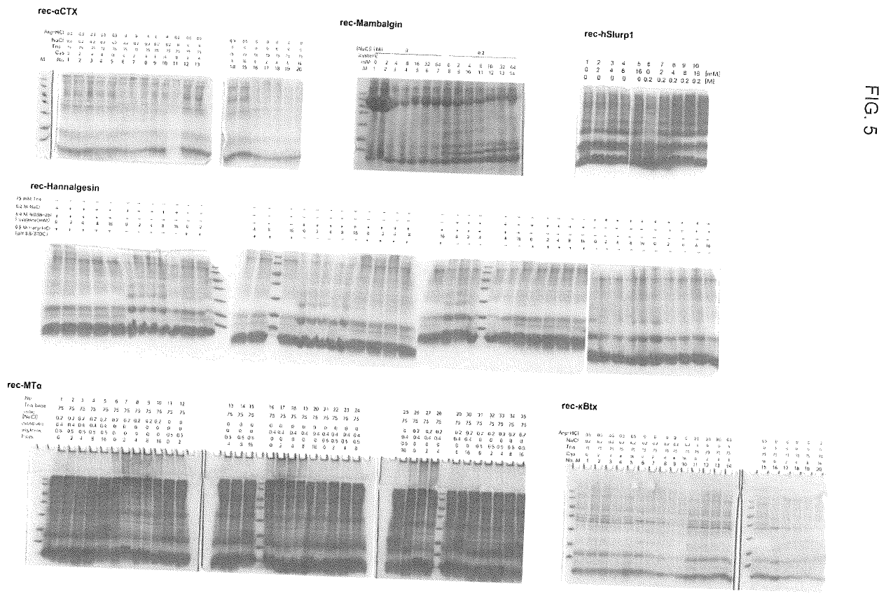

For optimized refolding condition for each recombinant neurotoxin, the most critical factors are the concentration of sodium chloride and l-cysteine and pH. L-arginine (see for example, Arakawa et al., 2007; Chen et al., 2008; Tischer et al., 2010; Tsumoto et al., 2004) and NDSB-201 (Luca et al., 2012; Wangkanont et al., 2015), two known supplements which are widely used in inclusion body refolding, even though significantly increased the yield of monomeric species in the screening experiment as reflected by non-reducing SDS-PAGE (FIG. 5), caused formation of a lot of precipitates in the subsequent dialysis removal of these supplements (data not shown), and thus actually resulted in lower yield.

In addition, recombinant three-fingered proteins (rTFPs) refolded with l-arginine usually were hard to crystallize (data not shown). What's more, l-arginine and NDSB-201 are very expensive and not cost-effective in large scale production. Taken together, L-arginine and NDSB-201 are generally not helpful for refolding rTFPs, at least for the rTFPs we attempted.

Normally, rTFPs with high isoelectric point (pI) remained soluble upon challenge with weak acidic solution (such as 20 mM NaAc, pH 5.0), while certain mammalian toxin-like protein, such as Slurp1, remained soluble only in neutral and slight basic solutions. Usually, if the refolded product remains soluble after the dialysis step, and does not contain species with significant inter-chain disulfide bond, as judged by existence of multimeric species on non-reducing SDS-PAGE, it is highly possible that the refolding is successful.

Complete Oxidation is the Key for High Quality rTFPs

It is common to see I.B. refolding protocols in which people dissolve the I.B. with solutions containing high concentration of reducing agents (such as 100 mM β-mercaptoethanol or 2-ME). While these agents are useful in keeping the free cysteine residue in reduced form and it might not be a problem in certain cases, we found 100 mM 2-ME in I.B. solubilization buffers inevitably lead to failed refolding experiments, which was shown by the extremely low yield and formation of multimeric species (Xu et al., 2015), thus should be avoided when solubilizing the I.B. For correct disulfide bonds pairing between the cysteine residues, a classical and widely used approach is the disulfide shuffling or mixed disulfide bond reactions, in which a predefined redox pairs such as a fixed ratio of reduced-glutathione:oxidized-glutathione, or cysteine:cystine are used (“Disulfide bond formation in proteins,” 1984; Okumura et al., 2011; Qin et al., 2015).

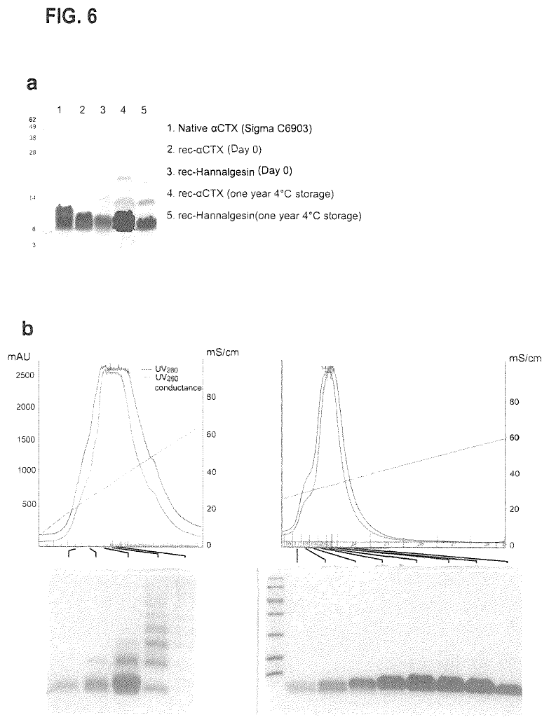

In our pipeline, we used a simple, straightforward approach by scouting cysteine concentration in screening refolding conditions, and we noticed that different recombinant three-fingered proteins (rTFPs) had different sensitivity to cysteine concentration in the refolding experiment, see FIG. 5. In preparative refolding we used compressed N₂ gas and/or air to drive the ultrafiltration device (see for example, FIG. 2). In refolding of rec-kBtx, we found N₂ gas was not as good as compressed air, which dramatically decreased the multimeric species in the final product, and only the purified rec-kBtx from this special protocol yielded crystals (see the following section). Clearly, ultrafiltration with the stirred cell is not only a physical process, but also a biochemical process in which dissolved oxygen level is critical for the correct and complete formation of disulfide bonds. A typical preparative ultrafiltration procedure took about 2 to 4 weeks, during the last a few days of which a large amount of white precipitate (which turned out to be cystine, the oxidized form of cysteine) showed up in the concentrated solution, which were found to be a good sign of complete oxidation, since most of our high quality rTFP were produced in this way. Some of our rTFPs were tested for their stability by prolonged storage at 4° C., and were shown to be ultra-stable even after one year of storage at 4° C., and only trace amounts of dimeric and multimeric species were found, see FIG. 6A. These observations were consistent to previous report about the stability of TFNs (Nirthanan et al., 2015)

### Concentrate to Dry is a Critical and Efficient Step for Removing Incorrectly Folded Species

It is interesting to note this point, since we found that multimeric species, which were generally regarded as incorrectly folded product with wrong pairing of disulfide bonds, were always present in the refolding product and hard to be separated from the correctly folded species using chromatography approaches, such as gel filtration and ion exchange. However, it turned out that ultrafiltration of the refolded product to dry dramatically increased the purity for some rTFPs, see FIG. 6B. It is thus noteworthy to try two ultrafiltration strategies, “to dry, or not to dry”, which in most cases could make a great difference.

Recombinant rTFPs Shows Good, Unique Behavior in Gel Filtration Chromatography and Biochemical Assays

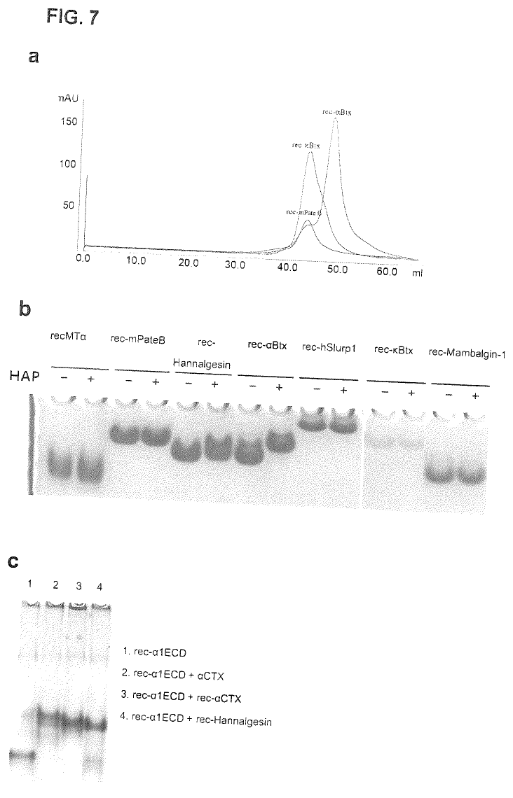

We compared the behavior of rec-αBtx, rec-κBtx, rec-mPate B in gel filtration column (SUPERDEX 75 10 300 GL™, GE Healthcare), and found that rec-αBtx behave like a monomer, while rec-κBtx behave like a dimer, see FIG. 7A, which is in accordance with earlier reports that κBtx exists in dimeric form and also to the solved structures (see the following sections). Rec-mPate B, also behave like a dimer,

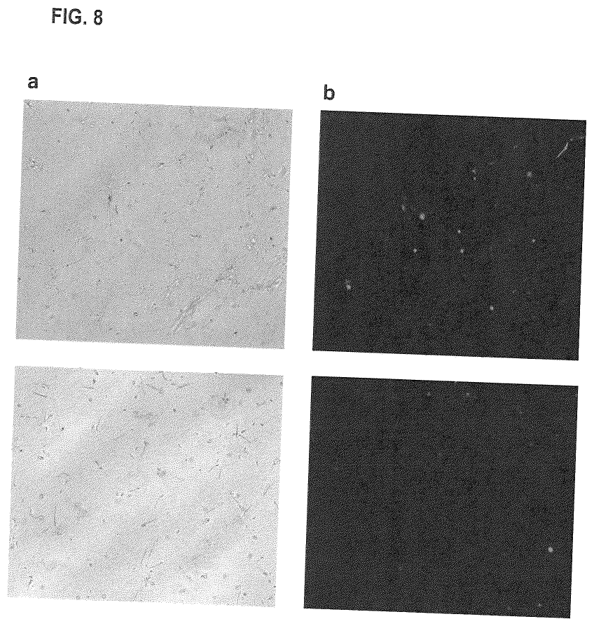

To test the binding specificities of the recombinant three-fingered proteins (rTFPs), HAP peptide, a known peptide derived from the nicotinic acetylcholine receptor (Harel et al., 2001), was mixed with various rTFPs and separated on a native PAGE gel at pH 5.0. HAP peptide was only able to shift rec-αBungarotoxin and only slightly shift rec-Hannalgesin, but not rec-MTα, rec-mPate B, rec-κBtx, and rec-hSlurp1, see FIG. 7B. Also, rec-αCTX and rec-Hannalgesin was shown to bind the extracellular domain of al subunit of the nicotinic acetylcholine receptor (α1ECD), like native αCTX isolated from Naja Kaouthia, see FIG. 7C. To test the possible binding activity of rec-mPate B to sperm, we labeled rec-mPate B with NHS-rhodamine and visualized the binding of rec-mPate B to spermatozoa freshly isolated from the epididymis of the mouse under the fluorescence microscope. The preliminary result did show binding of rec-mPate B to the head and tail of mouse spermatozoa, see FIG. 8A-B.

Structural Comparison of rTFPs Shows Almost the Same Structure as their Native Counterparts

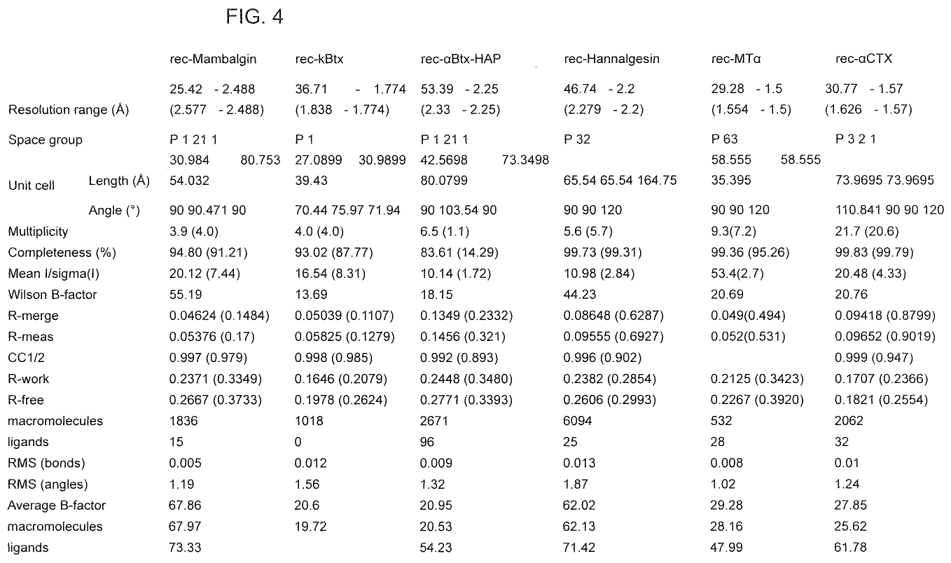

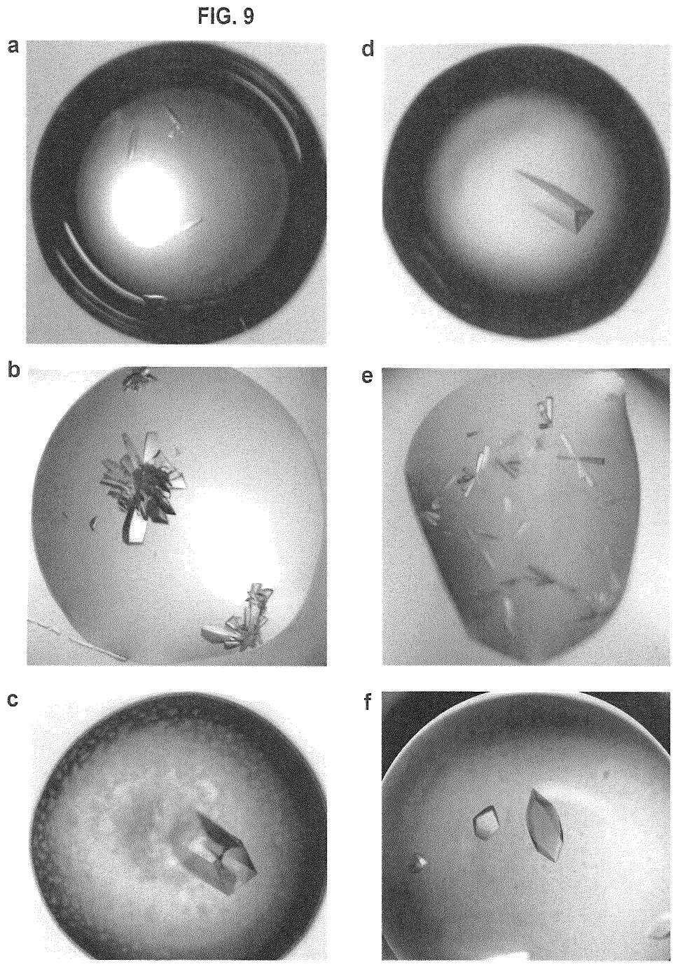

Most of our recombinant toxin crystals were formed at very high protein concentrations. They were beautiful-looking under polarized light under the microscope, see FIG. 9A-F, and diffracted x-ray quite well. Six (6) recombinant three-fingered proteins' (rTFPs') structures were solved with x-ray crystal diffraction data using molecular replacement with known homologous structures. The statistics for the data and structures were summarized in Table 1 (as illustrated in FIG. 4):

From the structural alignment of the solved structures with their native counterparts, such as rec-αBtx-HAP complex, rec-αCTX, rec-kBtx, rec-mambalgin, or with their most homologous native counterparts (such as rec-Hannalgesin and rec-MTα, whose crystal structure were not reported, known structure of αCTX and MT1, respectively, were used as the alignment counterpart), it is clear that our rTFPs are almost identical to their natural counterparts, except one or two amino acids at the N-terminal, which is a unique mark for their recombinant origin; as illustrated in FIG. 3.

### Discussion

Three-fingered proteins' (rTFPs) are a large collection of proteins (peptides) with important functions and applications. Traditionally, such proteins were isolated from the venom of the snakes, with very few recombinantly obtained in the lab with in depth analysis and verification. Because of their scarcity and unique properties and applications, these proteins are very expensive (at the level of hundreds to thousands of US dollars per milligrams) and some are not commercially available. κBtx, for example, a unique α₃β₂ nicotinic acetylcholine receptor binder, is not commercially available (personal communications). Because TFPs usually contain 4 to 5 pairs of disulfide bonds, it is usually very hard to recombinantly express them, and those commercially available are mostly purified from snake venoms. Some researchers used chemical synthesis that successfully obtained these rTFPs, such as mambalgin-1 and mambalgin-2 (Diochot et al., 2012; Mourier et al., 2016; Pan et al., 2014; Salinas et al., 2021; Schroeder et al., 2014; Sun et al., 2018). However, due to the high cost in chemical synthesis and limited yields, these successful attempts did not change the overall scenario for production of TFPs.

With our pipeline, however, milligrams to hundreds of milligrams of rTFPs could be obtained in the lab. Through extensive biochemical assays and structural analysis, we were able to show our rTFPs are almost identical to their native counterparts. Considering the fact that several of our rTFPs reached milligrams to hundreds of milligrams on a single lab-scale production cycle, these rTFP could thus replace their natural counterparts, and the method worth to be exploited for production of other TFPs further, which could be of general interest in the field.

### Legends

FIG. 1 illustrates a flow chart of an exemplary method as provided herein.

FIG. 2. SDS-PAGE analysis of rTFPs at different stages of production. con: control, not induced E. coli cells; pb: IPTG induced E. coli cells; I.B.: isolated inclusion bodies; purif.: purified final product (in non-reducing SDS-PAGE).

FIG. 3. Structural alignment of crystal structure of rTFPs and their natural counterpart or most homologous natural counterpart in Pymol. Yellow: rTFP, Magenta: Reported native or synthetic counterpart. a. rec-αBtx-HAP vs αBtx-HAP; b.

FIG. 5 illustrates a non-reducing SDS-PAGE image, illustrating refolding condition screening of rTNFs; refolding product from each refolding condition were concentrated and analyzed by 15% non-reducing SDS-PAGE.

FIG. 6A-B illustrates: FIG. 6A illustrates a non-reducing SDS-PAGE analysis of rec-αCTX and rec-Hannalgesin at different time points. FIG. 6B illustrates compared with “Not concentrated to dry” (left column), “Concentrate to dry” strategy (right column) dramatically increased the purity of the refolded rec-Hannalgesin, as shown by the non-reducing SDS-PAGE result of different fractions from cation exchange chromatography.

FIG. 7A-C illustrates: FIG. 7A illustrates a gel filtration analysis of rec-αBtx, rec-mPate B and rec-κBtx. FIG. 7B illustrates a native gel shift assay of various rTFPs with HAP peptide. FIG. 7C illustrates a native gel shift assay of α1ECD with native αCTX, rec-αCTX and rec-Hannalgesin.

FIG. 8A-B illustrates microscopic FIG. 8A and fluorescence microscopic FIG. 8B picture showing the binding of rec-mPate B to the spermatozoa from mouse epididymis.

FIG. 9 illustrates microscopic views of protein crystals from various rTFPs. a. rec-αBtx-HAP complex; b. rec-κBtx; c. rec-Mambalgin; d. rec-Hannalgesin; e. rec-MTα; f. rec-αCTX.

TABLE 2

| | | | No. of | | |
|---|---|---|---|---|---|
| | | | disulfide | Known | |
| TFP name | Origin | Specificity | bonds | structure? | Reference |
| αBtx | Bungarus | α₂βγε nAChR | 5 | Yes | (Chang and |
| | Multicinctus | blocker | | | Lee, 1963; |
| | | | | | Harel et al., |
| | | | | | 2001; Love |
| | | | | | and Stroud, |
| | | | | | 1986) |
| κBtx | Bungarus | α₃B₂ nAChR | 5 | Yes | (Cartier et al., |
| | Multicinctus | blocker | | | 1996; |
| | | | | | Chiappinelli, |
| | | | | | 1983; Dewan |
| | | | | | et al., 1994; |
| | | | | | Fiordalisi et |
| | | | | | al., 1994, |
| | | | | | 1991; Loring |
| | | | | | and Zigmond, |
| | | | | | 1988; Luetje et |
| | | | | | al., 1990) |
| Hannalgesin | Ophiophagus | NOS activator | 5 | No | (Pu et al., |
| | hannah | α₂βγε nAChR blocker | | | 1995a, 1995b) |
| Mambalgin-1 | Dendroaspis | ASIC1a blocker | 4 | Yes | (Diochot et al., |
| | polylepis | | | | 2012; Mourier |
| | | | | | et al., 2016; |
| | | | | | Salinas et al., |
| | | | | | 2021, 2014; |
| | | | | | Sun et al., |
| | | | | | 2018) |
| MTa | Dendrosaspis | a2B-adrenoceptor | 4 | No | (Koivula et al., |
| | angusticeps | blocker | | | 2010) |
| αCTX | Naja | α₂βγε nAChR | 4 | Yes | |
| | kaouthia | blocker | | | |
| mouse | Mouse | phosphatidylethanolamine | 5 | No | (Levitin et al., |
| Pate B | | phosphatidylserine | | | 2008; Luo et |
| | | | | | al., 2001; |
| | | | | | Turunen et al., |
| | | | | | 2011) |
| hSlurp1 | Human | α7 nAChR? | 5 | No | (Grønlien et |
| | | | | | al., 2007; |
| | | | | | Throm et al., |
| | | | | | 2018) |
| mSlurp1 | Mouse | | | | (Swamynathan |
| | | | | | et al., 2012; |
| | | | | | Upadhyay, 2019) |

rec-αBtx (V31)
Coding sequence:

(SEQ ID NO: 1)
atgggtATTGTCTGTCACACTACGGCAACGAGTCCGATCAGCGCA
GTTACGTGCCCGCCGGGTGAAAACCTGTGTTATCGTAAAATGTGG
TGCGATGTGTTTTGTAGCTCTCGCGGTAAAGTGGTTGAACTGGGT
TGCGCAGCAACCTGTCCGAGCAAAAAACCGTACGAAGAAGTTACC
TGCTGTTCTACGGATAAATGTAATCCGCATCCGAAACAGCGTCCG
GGTTAA

Translated protein sequence:

(SEQ ID NO: 2)
MGIVCHTTATSPISAVTCPPGENLCYRKMWCDVFCSSRGKVVELG
CAATCPSKKPYEEVTCCSTDKCNPHPKQRPG

Protein parameter: Number of amino acids: 76; Molecular weight: 8210.54; Theoretical pI: 8.36
Refolding result: good
I.B. solubilization solution: 50 mM Tris-HCl, pH 8.8, 8 M urea, 10 mM 2-ME
Refolding solution: 50 mM Tris-HCl, 16 mM l-cysteine, 0.2 M NaCl.
Key process: 1. Purify I.B. with Q Sepharose FF before refolding, and buffer exchanged to 50 mM Tris-HCl, pH 8.8, 8 M urea before refolding. 2. Ultrafiltrate to dry.
Yield: 0.05 mg/g bacteria (wet pellet)
Crystallization condition: mono S 5/50GL column purified rec-αBtx-HAP complex, buffer exchanged by dialysis to 0.1 M HEPES (pH 7.5), OD₂₈₀=11.04. 1:1 with 0.1 M HEPES (pH 7.5), 35% PEG3350, 0.2 M MgCl₂. 18° C.
X-ray diffraction data collection: yes
Structural solution: yes
   rec-αCTX
Coding sequence:

(SEQ ID NO: 3)
atgATCCGTTGCTTCATCACCCCGGACATCACCTCTAAAGACTGC
CCGAATGGCCACGTCTGCTACACGAAAACCTGGTGCGACGCTTTC
TGCTCTATCCGTGGTAAACGTGTTGACCTGGGTTGCGCTGCTACC
TGCCCGACCGTTAAAACCGGTGTTGACATCCAGTGCTGCTCTACC
GACAACTGCAACCCGTTCCCGACCCGTAAACGTCCGTAA

Translated protein sequence:

(SEQ ID NO: 4)
MIRCFITPDITSKDCPNGHVCYTKTWCDAFCSIRGKRVDLGCAAT
CPTVKTGVDIQCCSTDNCNPFPTRKRP

Protein parameter: number of amino acids: 72; Molecular weight: 7962.2; Theoretical pI: 8.59
Refolding result: good
I.B. solubilization solution: 50 mM Tris-HCl, pH 8.8, 6 M Guanidine-HCl, 5 mM 2-ME.
Refolding solution: 75 mM Tris base, 8 mM cysteine, 0.2 M NaCl.
key process: 1. Ultrafiltrate to dry. 2. Oxidization should be complete for emergence of large amount of white cystine crystalline.
Yield: 1 mg/g bacteria (wet pellet)
Crystallization condition: 110 mg/ml in 200 mM NH₄Ac (pH 7.0/25° C.), 1:1 with 0.1 M HEPES (pH 7.9), 30% Jeffamine M-600 (pH 7.0), 18° C.
X-ray diffraction data collection: yes
Structural solution: yes
   rec-κBtx:
coding sequence:

(SEQ ID NO: 5)
atgCGTACCTGTCTGATTAGCCCGTCCAGCACCCCGCAAACCTGT
CCGAATGGTCAAGATATTTGTTTTCTGAAGGCCCAGTGTGATAAA
TTTTGCAGCATTCGTGGCCCGGTGATCGAACAGGGTTGCGTTGCG
ACCTGTCCGCAATTTCGCTCTAACTACCGTTCACTGCTGTGCTGT
ACCACCGACAACTGTAATCATTAA

Translated protein sequence:

(SEQ ID NO: 6)
MRTCLISPSSTPQTCPNGQDICFLKAQCDKFCSIRGPVIEQGCVA
TCPQFRSNYRSLLCCTTDNCNH

Number of amino acids: 67; Molecular weight: 7406.5; Theoretical pI: 8.07

Refolding Result: good
I.B. solubilization solution: 50 mM Tris-HCl, pH 8.8, 8 M urea, 10 mM 2-ME
Refolding solution: 75 mM Tris-HCl, 16 mM cysteine, 0.2 M NaCl.
Key process:
1. Use compressed air to drive the Amicon Stirred Cell (200 ml).
2. Freezing and thawing the inclusion body solution (in 50 mM Tris-HCl, pH 8.8, 8 M urea, 10 mM 2-ME) and centrifugation to get rid of contaminating protein before refolding.
3. Ultrafiltrate to dry.
Yield: 0.1 mg purified product/g bacteria (wet pellet)
Crystallization condition: 117 mg/ml in 200 mM NH₄Ac, pH 7.0, 1:1 with 0.15 M DL-malic acid pH 7.0, 20% PEG 3350 at 4° C.
X-ray diffraction data collection: yes
Structural solution: yes
   rec-MTα
Coding sequence:

(SEQ ID NO: 7)
atgCTGACCTGCGTTACCTCCAAATCTATCTTCGGCATCACGACG
GAAAACTGCCCGGACGGCCAGAACCTGTGCTTCAAAAAGTGGTAT
TATCTGAACCATCGTTACAGCGATATTACGTGGGGTTGCGCAGCA
ACCTGTCCGAAACCGACGAACGTGCGCGAAACCATCCACTGCTGT
GAAACCGACAAGTGCAATGAATAA

Translated protein sequence:
MLTCVTSKSIFGITTENCPDGQNLCFKKWYYLNHRYSDITWGCAATCPKPTN VRETIHCCETDKCNE (SEQ ID NO:8)
Number of amino acids: 67 Molecular weight: 7684.7 Theoretical pI: 6.68.
I.B. solubilization solution: 50 mM Tris-HCl, pH 8.8, 6 M Guanidine-HCl, 5 mM 2-ME
Refolding result: good
refolding solution: 75 mM Tris base, 8 mM cysteine, 0.2 M NaCl.
key process: No.
Yield: approximately 1 to 2 mg purified product/g bacteria (wet pellet)
Crystallization condition: 128 mg/ml in 200 mM NH₄Ac (pH 7.0), 1:1 (v/v) with 1.26 M sodium phosphate monobasic monohydrate, 0.14 M potassium phosphate, pH 5.6 at 18° C.
X-ray diffraction data collection: yes
Structural solution: yes
   rec-Mambalgin-1
coding sequence:

(SEQ ID NO: 9)
atgAAGAGAGAAGCTGAAGCCTTAAAGTGCTATCAACACGGTAAA
GTCGTAACCTGCCACAGAGACATGAAGTTCTGCTATCACAACACA
GGTATGCCTTTTAGAAATTTGAAGTTGATATTGCAAGGTTGTTCT
TCATCCTGCTCTGAAACTGAAAACAATAAGTGCTGCTCCACCGAC
AGATGTAACAAAGGTTCA

Translated protein sequence:

MKREAEALKCYQHGKVVTCHRDMKFCYHNTGMPFRNLKLILQGCS
SSCSETENNKCCSTDRCNKGS

Number of amino acids: 66; Molecular weight: 7522.65; Theoretical pI: 8.87
Refolding result: good
I.B. solubilization solution: 50 mM Tris-HCl, pH 9.0, 6 M Guanidine-HCl, 5 mM 2-ME
Refolding solution: 50 mM Tris base, 16 mM cysteine, 0.2 M NaCl
Yield: approximately 0.2 to 0.5 mg purified product/g bacteria (wet pellet)
Key process: NOT concentrate to dry (would be hard to resolubilize if concentrate to dry), instead, dialyze the retention against 20 mM sodium acetate, pH 5.0 to remove contaminated proteins and multimeric species, which would precipitate out
Crystallization condition: rec-Mambalgin-1 (122 mg/ml in 200 mM NH4Ac (pH 7.0)), 1:1 (v/v) with 0.1 M HEPES, pH 7.0, 32% Jeffamine M600, 0.1 M KSCN, 18° C.
X-ray diffraction data collection: yes
Structural solution: yes
   rec-Hannalgesin
Coding sequence:

(SEQ ID NO: 10)
atgACGAAATGCTACGTTACCCCGGATGTTAAAAGCGAAACCTGC
CCGGCTGGTCAAGATATTTGCTACACGGAAACCTGGTGCGATGCG
TGGTGCACCAGCCGTGGCAAACGCGTCAACCTGGGTTGCGCGGCC
ACGTGTCCGATTGTGAAACCGGGCGTTGAAATCAAATGCTGCTCC
ACCGACAACTGTAACCCGTTCCCGACCCGCAAACGCCCGTAA

Translated protein sequence:

(SEQ ID NO: 11)
MTKCYVTPDVKSETCPAGQDICYTETWCDAWCTSRGKRVNLGCAA
TCPIVKPGVEIKCCSTDNCNPFPTRKRP

Number of amino acids: 73 Molecular weight: 8050.3 Theoretical pI: 8.36 Refolding result: good
I.B. solubilization solution: 50 mM Tris-HCl, pH 9.0, 6 M Guanidine-HCl, 5 mM 2-ME
Refolding solution: 75 mM Tris base, 16 mM cysteine, 0.2 M NaCl
Yield: approximately 1 to 2 mg purified product/g bacteria (wet pellet)
Key process: Concentrate to dry (would efficiently remove contaminated proteins and multimeric species) and resolubilize the peptide with 20 mM sodium acetate, pH 5.0
Crystallization condition: rec-hannalgesin in 80 mg/ml in 200 mM NH₄Ac (pH 7.0), 1:1 with 0.1 M Bis-Tris, 0.2 M (NH₄)₂SO₄, 25% PEG3350, at 18° C.
X-ray diffraction data collection: yes
Structural solution: yes

### Rec-mSlurp1 (Recombinant Mouse Slurp1)

Coding sequence:

(SEQ ID NO: 12)
atgTTTCGCTGCTATACCTGTGAACAACCGACGGCTATCAACTCA
TGTAAAAATATCGCTCAATGTAAAATGGAAGACACCGCCTGCAAA
ACCGTGCTGGAAACGGTTGAAGCGGCCTTTCCGTTCAACCATTCC
CCGATGGTCACCCGTAGCTGCAGCTCTAGTTGTCTGGCAACGGAT
CCGGACGGCATTGGTGTTGCGCACCCGGTGTTCTGCTGTTTCCGT
GACCTGTGTAACTCTGGTTTTCCGGGCTTTGTGGCGGGCCTGTAA

Translated protein sequence:

(SEQ ID NO: 13)
MFRCYTCEQPTAINSCKNIAQCKMEDTACKTVLETVEAAFPFNHS
PMVTRSCSSSCLATDPDGIGVAHPVFCCFRDLCNSGFPGFVAGL

Number of amino acids: 89; Molecular weight: 9594.04; Theoretical pI: 5.47
Refolding result: good
I.B. solubilization solution: 50 mM Tris base, 8 M urea, 5 mM 2-ME
Refolding condition: 50 mM Tris-HCl (pH 9.0), 4 mM cysteine
Key process: concentrate NOT to dry, dialyze against 10 mM HEPES, pH 7.5 and purified with mono Q column
Crystallization condition: 128 mg/ml with equal 0.8 M Succinic acid pH 7.0 at 4° C.
X-ray diffraction data collection: No

### Rec-hSlurp1 (Recombinant Human Slurp1)

coding sequence:

(SEQ ID NO: 14)
atgCTGAAATGCTACACCTGCAAAGAACCGATGACCTCTGCTTCT
TGCCGTACCATCACCCGTTGCAAACCGGAAGACACCGCTTGCATG
ACCACCCTGGTTACCGTTGAAGCTGAATACCCGTTCAACCAGTCT
CCGGTTGTTACCCGTTCTTGCTCTTCTTCTTGCGTTGCTACCGAC
CCGGACTCTATCGGTGCTGCTCACCTGATCTTCTGCTGCTTCCGT
GACCTGTGCAACTCTGAACTGTAA

Translated protein sequence:

(SEQ ID NO: 15)
MLKCYTCKEPMTSASCRTITRCKPEDTACMTTLVTVEAEYPFNQS
PVVTRSCSSSCVATDPDSIGAAHLIFCCFRDLCNSEL

Number of amino acids: 82; Molecular weight: 8984.3; Theoretical pI: 5.15
Refolding Result: good
I.B. solubilization solution: 50 mM Tris base, 8 M urea, 5 mM 2-ME
Refolding condition: 50 mM Tris-HCl, 0.2 M NaCl, 4 mM cysteine
Key process: 1. Fusion of 6× Histidine tag would significantly reduce refolding efficiency; 2. NOT concentrate to dry after refolding, dialyze against 10 mM HEPES, pH 7.5 and purified with mono Q column
Crystallization condition: no crystal obtained

### Rec-mPate-B (Recombinant Mouse Pate B)

Coding sequence:

(SEQ ID NO: 16)
atgCTGATCTGCAACTCTTGCGAAAAATCTCGTGACTCTCGTTGC
ACCATGTCTCAGTCTCGTTGCGTTGCTAAACCGGGTGAATCTTGC
TCTACCGTTTCTCACTTCGTTGGTACCAAACACGTTTACTCTAAA
CAGATGTGCTCTCCGCAGTGCAAAGAAAAACAGCTGAACACCGGT
AAAAAACTGATCTACATCATGTTCGGTGAAAAAAACCTGATGAAC
TTCctcgagCACCACCACCACCACCACTGA

Translated protein sequence:

(SEQ ID NO: 17)
MLICNSCEKSRDSRCTMSQSRCVAKPGESCSTVSHFVGTKHVYSK
QMCSPQCKEKQLNTGKKLIYIMFGEKNLMNFLEHHHHHH

Number of amino acids: 84; Molecular weight: 9683.22; Theoretical pI: 9.08
Refolding Result: good
I.B. solubilization solution: 5 mM imidazole, 6 mM Guanidine-HCl, 10 mM 2-ME
Refolding condition: 50 mM Tris-HCl, pH 9Key process: 1. Fusion of 6×Histidine tag did not significantly reduce refolding efficiency; 2. Concentrating to dry after refolding would efficiently remove contaminated proteins and multimeric species) and resolubilize the peptide with 20 mM sodium acetate, pH 5.0 and purified with mono S column
Crystallization condition: no crystal obtained

### REFERENCES

Akbar A, et al. 2019. BMC Med Genet 20:145.
Antil-Delbeke S, et al. 2000. J Biol Chem 275:29594-29601.
Arakawa T, et al. 2007. Biophys Chem 127:1-8.
Battye T G G, et al. 2011. Acta Crystallogr D Biol Crystallogr 67:271-281.
Cartier G E, et al., J Biol Chem 271:7522-7528.
Chang C C, Lee C Y. 1963. Arch Int Pharmacodyn Ther 144:241-257.
Chen J, et al. 2008. Biotechnol Prog 24:1365-1372.
Chiappinelli V A. 1983. Brain Res 277:9-22.
Chiappinelli V A, Hue B, Mony L, Sattelle D B. 1989. J Exp Biol 141:61-71.
Dellisanti C D, et al. 2007. Nat Neurosci 10:953-962.
Dewan J C, et al. 1994. Biochemistry 33:13147-13154.
Diochot S, et al. 2012. Nature 490:552-555.
Disulfide bond formation in proteins. 1984. Methods in Enzymology. Academic Press. pp. 305-329.
Faure G, et al. 2016. Dokl Biochem Biophys 468:193-196.
Fiala G J, et al. 2011. J Vis Exp. doi: 10.3791/2164
Fiordalisi J J, et al. 1994. Affinity of native kappa-bungarotoxin and site-directed mutants for the muscle nicotinic acetylcholine receptor. Biochemistry 33:12962-12967.
Fiordalisi J J, et al. 1991. Synthesis and expression in Escherichia coli of a gene for kappa-bungarotoxin. Biochemistry 30:10337-10343.
Fiordalisi J J, et al. 1996. Toxicon 34:213-224.
Fischer J, Bouadjar et al. 2001. Mutations in the gene encoding SLURP-1 in Mal de Meleda. Hum Mol Genet 10:875-880.
Fruchart-Gaillard C, et al. 2012. Engineering of three-finger fold toxins creates ligands with original pharmacological profiles for muscarinic and adrenergic receptors. PLoS One 7:e39166.
George A A, et al. 2017. Isoform-specific mechanisms of α3β4*-nicotinic acetylcholine receptor modulation by the prototoxin lynx1. FASEB J 31:1398-1420.
Grønlien J H, et al. 2007. Distinct profiles of alpha7 nAChR positive allosteric modulation revealed by structurally diverse chemotypes. Mol Pharmacol 72:715-724.
Harel M, et al. 2001. The binding site of acetylcholine receptor as visualized in the X-Ray structure of a complex between alpha-bungarotoxin and a mimotope peptide. Neuron 32:265-275.
Huang Y, et al. 2006. Defining the CD59-C9 binding interaction. J Biol Chem 281:27398-27404.
Kieffer B, et al. 1994. Three-dimensional solution structure of the extracellular region of the complement regulatory protein CD59, a new cell-surface protein domain related to snake venom neurotoxins. Biochemistry 33:4471-4482.
Koivula K, et al. 2010. The three-finger toxin MTalpha is a selective alpha(2B)-adrenoceptor antagonist. Toxicon 56:440-447.
Krajewski J L, Dickerson I M, Potter L T. 2001. Site-directed mutagenesis of m1-toxin1: two amino acids responsible for stable toxin binding to M(1) muscarinic receptors. Mol Pharmacol 60:725-731.
Kudryavtsev D S, et al. 2020. Complex approach for analysis of snake venom α-neurotoxins binding to HAP, the high-affinity peptide. Sci Rep 10:3861.
Levandoski et al. 2000. Recombinant expression of alpha-bungarotoxin in Pichia pastoris facilitates identification of mutant toxins engineered to recognize neuronal nicotinic acetylcholine receptors. J Neurochem 74:1279-1289.
Levitin F, et al. 2008. PATE gene clusters code for multiple, secreted TFP/Ly-6/uPAR proteins that are expressed in reproductive and neuron-rich tissues and possess neuromodulatory activity. J Biol Chem 283:16928-16939.
Liebschner D, et al. 2019. Macromolecular structure determination using X-rays, neutrons and electrons: recent developments in Phenix. Acta Crystallogr D Struct Biol 75:861-877.
Loring R H, Zigmond R E. 1988. Characterization of neuronal nicotinic receptors by snake venom neurotoxins. Trends Neurosci 11:73-78.
Love R A, Stroud R M. 1986. The crystal structure of alpha-bungarotoxin at 2.5 A resolution: relation to solution structure and binding to acetylcholine receptor. Protein Eng 1:37-46.
Luca V C, AbiMansour J, Nelson C A, Fremont D H. 2012. Crystal structure of the Japanese encephalitis virus envelope protein. J Virol 86:2337-2346.
Luetje C W, et al. 1990. Neurotoxins distinguish between different neuronal nicotinic acetylcholine receptor subunit combinations. J Neurochem 55:632-640.
Luo C W, Lin H J, Chen Y H. 2001. A novel heat-labile phospholipid-binding protein, SVS VII, in mouse seminal vesicle as a sperm motility enhancer. J Biol Chem 276:6913-6921.
Lyukmanova E, et al. 2017. Lynx1 competes with A beta 1-42 for the binding to nicotinic acetylcholine receptors JOURNAL OF NEUROCHEMISTRY. WILEY 111 RIVER ST, HOBOKEN 07030-5774, NJ USA. pp. 215-215.
Lyukmanova E N, et al. 2016. Secreted Isoform of Human Lynx1 (SLURP-2): Spatial Structure and Pharmacology of Interactions with Different Types of Acetylcholine Receptors. Sci Rep 6:30698.
Margalit M, et al. 2012. Involvement of the prostate and testis expression (PATE)-like proteins in sperm-oocyte interaction. Hum Reprod 27:1238-1248.
Michielsen L A, et al. 2017. Reduced Expression of Membrane Complement Regulatory Protein CD59 on Leukocytes following Lung Transplantation. Front Immunol 8:2008.
Mourier G, et al. 2016. Mambalgin-1 Pain-relieving Peptide, Stepwise Solid-phase Synthesis, Crystal Structure, and Functional Domain for Acid-sensing Ion Channel 1a Inhibition. J Biol Chem 291:2616-2629.
Näreoja K, et al. 2011. Adrenoceptor activity of muscarinic toxins identified from mamba venoms. Br J Pharmacol 164:538-550.
Nevo Y, et al. 2013. CD59 deficiency is associated with chronic hemolysis and childhood relapsing immune-mediated polyneuropathy. Blood 121:129-135.
Nirthanan S, et al. 2015. Snake α-Neurotoxins and the Nicotinic Acetylcholine Receptor In: Gopalakrishnakone P, Inagaki H, Mukherjee A K, Rahmy T R, Vogel C-W, editors. Snake Venoms. Dordrecht: Springer Netherlands. pp. 1-39.
Okumura M, et al. 2011. Acceleration of disulfide-coupled protein folding using glutathione derivatives. FEBS J 278:1137-1144.
Otwinowski Z, Minor W. 1997. Processing of X-ray diffraction data collected in oscillation mode. Methods Enzymol 276:307-326.
Pan M, He Y, Wen M, Wu F, Sun D, Li S, Zhang L, Li Y, Tian C. 2014. One-pot hydrazide-based native chemical ligation for efficient chemical synthesis and structure determination of toxin Mambalgin-1. Chem Commun 50:5837-5839.
Pu X C, Gopalakrishnakone P, Wong P T H. 1995a. Neurotoxin CM-11 from king cobra venom (Ophiophagus hannah) causes antinociception without neurological deficit. Toxicon 33:719.
Pu X C, et al. 1995b. A novel analgesic toxin (hannalgesin) from the venom of king cobra (Ophiophagus hannah). Toxicon 33:1425-1431.
Qin M, Wang W, Thirumalai D. 2015. Protein folding guides disulfide bond formation. Proc Natl Acad Sci USA 112:11241-11246.
Rajesh A, et al. 2017. shRNA mediated ablation of prostate and testis expressed (Pate) messenger RNA results in impaired sperm function and fertility. Andrology 5:541-547.
Rosenthal J A, et al. 1994. Functional expression and site-directed mutagenesis of a synthetic gene for alpha-bungarotoxin. J Biol Chem 269:11178-11185.
Salinas M, et al. 2014. Binding site and inhibitory mechanism of the mambalgin-2 pain-relieving peptide on acid-sensing ion channel 1a. J Biol Chem 289:13363-13373.
Salinas M, et al. 2021. Mambalgin-1 pain-relieving peptide locks the hinge between α4 and α5 helices to inhibit rat acid-sensing ion channel 1a. Neuropharmacology 185:108453.
Schroeder C I, et al. 2014. Chemical synthesis, 3D structure, and ASIC binding site of the toxin mambalgin-2. Angew Chem Int Ed Engl 53:1017-1020.
Servent D, Fruchart-Gaillard C. 2009. Muscarinic toxins: tools for the study of the pharmacological and functional properties of muscarinic receptors. J Neurochem 109:1193-1202.
Shah K, et al. 2016. A novel homozygous mutation disrupting the initiation codon in the SLURP1 gene underlies mal de Meleda in a consanguineous family. Clin Exp Dermatol 41:675-679.
Sun D, et al. 2018. Cryo-EM structure of the ASIC1a-mambalgin-1 complex reveals that the peptide toxin mambalgin-1 inhibits acid-sensing ion channels through an unusual allosteric effect. Cell Discov 4:27.
Swamynathan S, et al. 2012. Klf4 regulates the expression of Slurp1, which functions as an immunomodulatory peptide in the mouse cornea. Invest Ophthalmol Vis Sci 53:8433-8446.
Swamynathan S, et al. 2016. Secreted Ly6/Urokinase-type Plasminogen activator Receptor-Related Protein-1 (SLURP1) Suppresses Angiogenic Inflammation through a Pathway that Involves Src, RhoA and NFκB. Invest Ophthalmol Vis Sci 57:1447-1447.
Taylor J A, et al. 2016. Mal de Meleda in Indonesia: Mutations in the SLURP1 gene appear to be ubiquitous. Australas J Dermatol 57: e11-3.
Thomsen M S, et al. 2016. Lynx1 and Aß1-42 bind competitively to multiple nicotinic acetylcholine receptor subtypes. Neurobiol Aging 46:13-21.
Throm V M, et al. 2018. Endogenous CHRNA7-ligand SLURP1 as a potential tumor suppressor and anti-nicotinic factor in pancreatic cancer. Oncotarget 9:11734-11751.
Tischer A, et al. 2010. L-arginine hydrochloride increases the solubility of folded and unfolded recombinant plasminogen activator rPA. Protein Sci 19:1783-1795.
Trémeau O, et al. 1995. Genetic engineering of snake toxins. The functional site of Erabutoxin a, as delineated by site-directed mutagenesis, includes variant residues. J Biol Chem 270:9362-9369.
Tsumoto K, et al. 2004. Role of arginine in protein refolding, solubilization, and purification. Biotechnol Prog 20:1301-1308.
Turunen H T, et al. 2011. Reprod Biol Endocrinol 9:128.
Upadhyay G. 2019. Front Immunol 10:819.
Wangkanont K, et al. 2015. Protein Expr Purif 115:19-25.
Winn M D, et al. 2011. Acta Crystallogr D Biol Crystallogr 67:235-242.
Wittig I, Schägger H. 2005. Proteomics 5:4338-4346.
Xu J, et al. 2015. Protein Expr Purif 110:30-36.
Yao Y, et al. 2002. J Biol Chem 277:12613-12621.

A number of embodiments of the invention have been described. Nevertheless, it can be understood that various modifications may be made without departing from the spirit and scope of the invention. Accordingly, other embodiments are within the scope of the following claims.

1: A method for producing and purifying a disulfide-linked protein, comprising:
(a) providing an isolated or substantially isolated bacterial inclusion body comprising a recombinantly expressed disulfide-linked protein or peptide recombinantly expressed in the bacteria;
(b) solubilizing the inclusion body in a solubilization buffer;
(c) oxidatively refolding the inclusion body in a refolding buffer comprising:
(i) incubating the solubilized inclusion body in the refolding buffer, and
(ii) concentrating the incubated refolding buffer comprising the solubilized inclusion body and recombinantly expressed disulfide-linked protein or peptide to generate a concentrated solution or dry powder comprising the recombinantly expressed disulfide-linked protein or peptide; and
(d) purifying or substantially purifying or substantially isolating the recombinantly expressed disulfide-linked protein or peptide by a method comprising:
(i) re-solubilizing or solubilizing the concentrated solution or dry powder comprising the recombinantly expressed disulfide-linked protein or peptide in a low ionic strength buffer before purifying or substantially purifying or substantially; and
(ii) purifying or substantially purifying or substantially isolating the recombinantly expressed disulfide-linked protein or peptide, optionally by a method comprising use of a chromotography.

2: The method of claim 1, wherein the disulfide-linked protein or peptide comprises or is: a three-finger neurotoxin peptide.

3: The method of claim 2, wherein the disulfide-linked protein comprises: a three-finger neurotoxin peptide comprises or is: recombinant (rec)-α-Bungarotoxin (rec-αBtx), rec-α-Cobratoxin (rec-αCTX), κ-Bungarotoxin (rec-κBtx), rec-MTa, rec-hannalgesin, or rec-Mambalgin, rec-Slurp, rec-Pate).

4: The method of claim 1, wherein the disulfide-linked protein or peptide comprises or is: an antibody, an antibody fragment, a single chain antibody, an extracellular domain of a viral membrane protein, a cell surface receptor, a dendrotoxin, or a conotoxin.

5: The method of claim 1, wherein the bacterial inclusion body comprises or is an inclusion body from a bacteria of the Escherichia genus, or is an E. coli inclusion body.

6: The method of claim 1, wherein the solubilizing comprises use of a solubilization buffer comprising urea and/or guanidine, or comprising 50 mM Tris-HCl (pH 8.0), 8 M urea or 6 M guanidine-HCl and 5 mM 2-Mercaptoethanol (2-ME); or, 50 mM Tris-HCl (pH 9.0), 6 M guanidine-HCl, 5 mM 2-ME.

7: A method for producing a disulfide-linked protein, comprising:
(a) providing an isolated or substantially isolated bacterial inclusion body comprising a recombinantly expressed disulfide-linked protein or peptide recombinantly expressed in the bacteria;
(b) solubilizing the inclusion body in a solubilization buffer; and
(c) oxidatively refolding the inclusion body in a refolding buffer comprising:
(i) incubating the solubilized inclusion body in the refolding buffer, and
(ii) concentrating the incubated refolding buffer comprising the solubilized inclusion body and recombinantly expressed disulfide-linked protein or peptide to generate a concentrated solution or dry powder comprising the recombinantly expressed disulfide-linked protein or peptide.

8: The method of claim 7, wherein the disulfide-linked protein or peptide comprises or is: a three-finger neurotoxin peptide.

9: The method of claim 8, wherein the disulfide-linked protein comprises: a three-finger neurotoxin peptide comprises or is: recombinant (rec)-α-Bungarotoxin (rec-αBtx), rec-α-Cobratoxin (rec-αCTX), κ-Bungarotoxin (rec-κBtx), rec-MTa, rec-hannalgesin, or rec-Mambalgin, rec-Slurp, rec-Pate).

10: The method of claim 7, wherein the disulfide-linked protein or peptide comprises or is: an antibody, an antibody fragment, a single chain antibody, an extracellular domain of a viral membrane protein, a cell surface receptor, a dendrotoxin, or a conotoxin.

11: The method of claim 7, wherein the bacterial inclusion body comprises or is an inclusion body from a bacteria of the Escherichia genus, or is an E. coli inclusion body.

12: The method of claim 7, wherein the solubilizing comprises use of a solubilization buffer comprising urea and/or guanidine, or comprising 50 mM Tris-HCl (pH 8.0), 8 M urea or 6 M guanidine-HCl and 5 mM 2-Mercaptoethanol (2-ME); or, 50 mM Tris-HCl (pH 9.0), 6 M guanidine-HCl, 5 mM 2-ME.

13: The method of claim 7, wherein the incubating comprises conditions comprising incubating for between about 1 to 24 hours, or for between about 6 to 12 hours.

14: The method of claim 7, wherein the incubating comprises conditions comprising a temperature of about 4° C. or an ice-cold solution, and/or incubating under pressure, optionally by use of compressed air or by use of a nitrogen tank or a reaction vessel.

15: The method of claim 7, wherein the refolding buffer is stirred during the incubating.

16: The method of claim 1, wherein oxidatively refolding the inclusion body in a refolding buffer comprises:
(a) incubating the solubilized inclusion body in the refolding buffer:
(i) for between about 1 to 24 hours, or for between about 6 to 12 hours,
(ii) under conditions comprising 4° C. or in an ice-cold solution, or
(iii) under pressure in a compressed air or nitrogen tank or reaction vessel, or
(b) stirring the refolding buffer during the incubating.

17: The method of claim 1, wherein oxidatively refolding the inclusion body in a refolding buffer comprises:
(a) concentrating using a nanofiltration device the incubated refolding buffer comprising the solubilized inclusion body and recombinantly expressed disulfide-linked protein or peptide to generate a concentrated solution,
(b) concentrating using a compressed nitrogen-gas (or air) driven ultrafiltration device the incubated refolding buffer comprising the solubilized inclusion body and recombinantly expressed disulfide-linked protein or peptide to generate a concentrated solution, or
(c) concentrating to about 1, 2, 3, 4 or 5 ml.

18: The method of claim 7, wherein oxidatively refolding the inclusion body in a refolding buffer comprises:
(a) concentrating using a nanofiltration device the incubated refolding buffer comprising the solubilized inclusion body and recombinantly expressed disulfide-linked protein or peptide to generate a concentrated solution,
(b) concentrating using a compressed nitrogen-gas (or air) driven ultrafiltration device the incubated refolding buffer comprising the solubilized inclusion body and recombinantly expressed disulfide-linked protein or peptide to generate a concentrated solution, or
(c) concentrating to about 1, 2, 3, 4 or 5 ml.
